(19) United States
(12) Patent Application Publication
(10) Pub. No.: US 2022/0183345 A1
(43) Pub. Date: Jun. 16, 2022

(54) Nicotine Chew Device for Denture Users

(71) Applicant: Mary Lavender, Cincinnati, OH (US)

(72) Inventor: Mary Lavender, Cincinnati, OH (US)

(21) Appl. No.: 17/485,604

(22) Filed: Sep. 27, 2021

Related U.S. Application Data
(60) Provisional application No. 63/124,147, filed on Dec. 11, 2020.

(51) Int. Cl. A24B 15/18; A24B 15/16

(52) U.S. Cl. CPC A24B 15/186; A24B 15/16

(57) ABSTRACT

The present invention relates generally to the field of chewing products. More specifically, the present invention relates to a nicotine chew device for denture users. The device can be used by a denture wearer in order to quit smoking by chewing the device to release nicotine. Further, the device is preferably comprised of a base, sweetener, softener/plasticizer agent, flavoring agent, coloring agent, and wax coating. In one embodiment, the chew may have a wax base and/or wax coating that does not stick to the surface of dentures. In various embodiments, the wax base and wax coating may be nicotine infused.

### CROSS-REFERENCE TO RELATED APPLICATION

The present application claims priority to, and the benefit of, U.S. Provisional Application No. 63/124,147, which was filed on Dec. 11, 2020 and is incorporated herein by reference in its entirety.

### FIELD OF THE INVENTION

The present invention relates generally to the field of chewing products. More specifically, the present invention relates to a nicotine chew device for denture users. The device can be used by a denture wearer in order to quit smoking by chewing the device to release nicotine. Further, the device is preferably comprised of a base, sweetener, softener/plasticizer agent, flavoring agent, coloring agent and wax coating. In one embodiment, the chew may have a wax base and/or wax coating that does not stick to the surface of dentures. In various embodiments, the wax base and wax coating may be nicotine infused. Accordingly, the present disclosure makes specific reference thereto. Nonetheless, it is to be appreciated that aspects of the present invention are also equally applicable to other like applications, devices and methods of manufacture.

### BACKGROUND

In 2019, 14 percent of U.S. adults aged 18 years or older (approximately 34.1 million adults) smoked cigarettes. Further, more than 16 million Americans live with a smoking-related disease. In order to quit smoking cigarettes, many individuals use nicotine patches, while others use nicotine gum. It is estimated that between 1.5-2 million Americans try nicotine gum every year in order to receive similar amounts of nicotine to that of a cigarette in order to quit smoking.

Further, some individuals who use nicotine gum to quit smoking wear dentures. However, sticky foods such as gum present a problem for denture wearers. Because of their consistency, sticky foods and gum can allow dentures to slip slightly while chewing. This allows food particles to get under the partial denture, which can cause sore spots on the gums of the denture wearer. This is especially true if the clasps of the denture are loose. Further, chewing gum can easily become wrapped around the clasps and under the partial of a denture and may dislodge the denture entirely. In addition, the gum may stick to the surface of the denture itself.

In order to remove chewing gum from dentures, a user must attempt to scrape away as much of the gum as possible. This is typically done by using a sharp object such as a knife, which could easily cause injury to the user if the knife slips off the surface of the dentures while scraping the gum. Other methods of removing gum from dentures involve soaking dentures in warm vinegar to dissolve the remaining gum residue. This is undesirable as it requires a user to take out their dentures, such that they cannot eat for a prolonged period of time. Further, some methods of gum removal involve heating dentures with a hairdryer to soften the gum. However, this can be extremely risky as excessive heat can damage dentures and lead to costly repairs.

Therefore, there exists a long-felt need in the art for a gum or other similar chewing device that can easily be chewed by users with dentures. Further, there exists a long-felt need in the art for a gum or other similar chewing device that aids a user in quitting smoking. Finally, there exists a long-felt need in the art for an improved nicotine gum or other similar chewing device that does not stick to the surface of dentures.

The subject matter disclosed and claimed herein, in one embodiment thereof, comprises a nicotine chew for denture users. The chew is comprised of a gum base, a sweetener, a softener/plasticizer agent, a flavoring agent, a coloring agent and a wax coating. Further, the base portion of the chew may be made of wax such that it does not stick to the surface of dentures. In addition, the chew or wax coating may be infused with nicotine, such that the chew allows a user to quit smoking in a similar manner to nicotine gum.

In this manner, the nicotine chew for denture users of the present invention accomplishes all of the forgoing objectives and provides a gum that can be chewed by individuals with dentures. Further, the chew can be used to aid users in quitting smoking. In addition, the chew provides a much needed alternative to existing nicotine gum, which sticks to dentures.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a nicotine chew device for denture users. The device is comprised of a nicotine-infused wax chew that can be chewed by denture users/wearers who wish to quit smoking. The chew is comprised of a gum base, a sweetener, a softener/plasticizer agent, a flavoring agent, a coloring agent and a wax coating. The base is preferably comprised of a chewable natural or synthetic substance. The sweetener is preferably natural and plant-derived, although the chew may contain other synthetic or organic sweeteners that are not plant-derived in differing embodiments. The softener/plasticizer agent further allows the chew to retain moisture and increase flexibility. The chew also has a wax coating that may also contain a variety of oils. Further, the chew may come in a plurality of flavors and colors. In differing embodiments, the chew may also be in a plurality of shapes and may be relatively two-dimensional (e.g. flat) or three-dimensional.

The present invention also discloses a method of applying wax coating to one potential embodiment of the chew. The method involves first dipping, brushing or spraying a wax coating onto the surface of the chew. Further, the coating may be infused with nicotine such that the chew releases nicotine when chewed. Finally, the chew may receive a powder dusting of polyol to extend the chew's shelf life. It should also be noted that in one embodiment, the chew is infused with nicotine before the wax coating is applied.

The chew may also be stored in a packaging that has individually separated compartments. Each compartment is further connected to another compartment via a perforation, such that each compartment can be torn from one another. Further, each compartment contains a peelable cover that seals the chew within the compartment until use.

Accordingly, the improved nicotine chew device for denture users of the present invention is particularly advantageous as it allows denture users to utilize a chewing product that does not damage or stick to their dentures. Further, the product allows denture users to safely use a nicotine chew device to quit smoking. As a result, denture users have the option to quit smoking with a nicotine chew instead of traditional nicotine gum or a nicotine patch.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a graphical view of essential components of one potential embodiment of a nicotine chew device for denture users of the present invention in accordance with the disclosed architecture;

FIG. 2 illustrates a graphical view of a method of applying a wax coating to one potential embodiment of a nicotine chew device for denture users of the present invention in accordance with the disclosed architecture;

FIG. 3 illustrates a bottom perspective view of one potential embodiment of a nicotine chew device for denture users of the present invention in packaging in accordance with the disclosed architecture; and

FIG. 4 illustrates a perspective view of one potential embodiment of a nicotine chew device for denture users of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a gum or similar chewing device that can be easily chewed by users with dentures. Further, there exists a long-felt need in the art for a gum or similar chewing device that aids a user in quitting smoking. Finally, there exists a long-felt need in the art for an improved nicotine gum or similar chewing device that does not stick to the surface of dentures.

The present invention, in one exemplary embodiment, is comprised of a nicotine chew device for denture users. The device is comprised of a nicotine-infused wax chew that can be chewed by denture wearers who wish to quit smoking. The chew is comprised of a gum base, a sweetener, a softener/plasticizer agent, a flavoring agent, a coloring agent and a wax coating. The base is preferably comprised of a chewable natural or synthetic substance. The sweetener is preferably natural and planted derived, although the chew may contain other synthetic or organic sweeteners that are not plant-derived. The softener/plasticizer agent further allows the chew to retain moisture and increase flexibility. The chew also has a wax coating that may also contain a variety of oils. Further, the chew may come in a plurality of flavors and colors, and may also be a plurality of shapes such as relatively two-dimensional (e.g. flat) or three-dimensional.

The present invention also discloses a method of applying wax coating to one potential embodiment of the chew that involves dipping, brushing or spraying a wax coating onto the surface of the chew. Then, the coating may be infused with nicotine such that the chew releases nicotine when chewed. However, it should be noted that the chew may be already infused with nicotine before any wax coating is applied. Finally, the chew may receive a powder dusting of polyol to extend the shelf life of the chew.

In addition, the chew may also be stored in a packaging that has individually separated compartments. Each compartment is further connected to another compartment via a perforation, such that each compartment can be torn from one another. Further, each compartment contains a peelable cover that seals the chew within the compartment.

Accordingly, the improved nicotine chew device for denture users of the present invention is particularly advantageous as it allows denture users to use a chewing product that does not damage or stick to their dentures. Further, the product allows denture users to safely use a nicotine chew device to quit smoking. As a result, denture users have the option to quit smoking with a nicotine chew instead of nicotine gum or a nicotine patch.

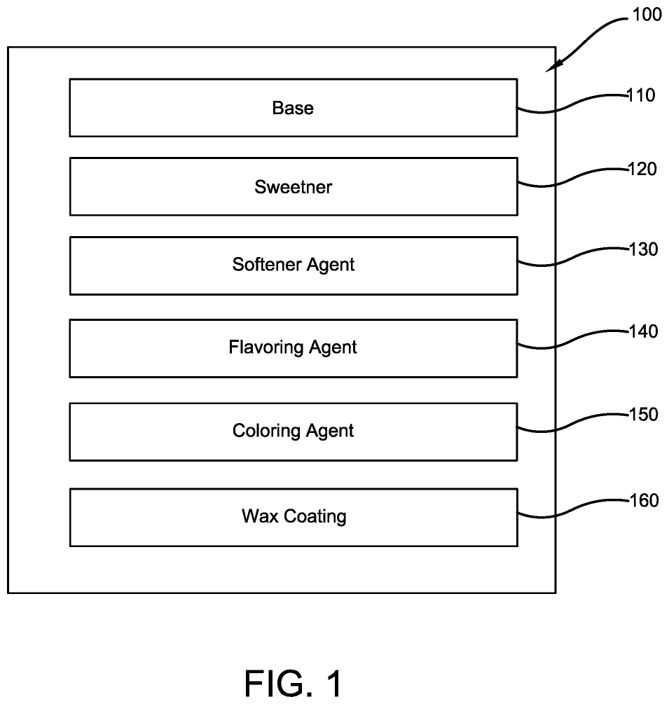

Referring initially to the drawings, FIG. 1 illustrates a graphical view of essential components of one potential embodiment of a nicotine chew for denture users 100 of the present invention in accordance with the disclosed architecture. The chew 100 is primarily comprised of a gum base 110, a sweetener 120, a softener/plasticizer agent 130, a flavoring agent 140, a coloring agent 150 and a wax coating 160. However, differing embodiments of the chew 100 may lack any of said components 110, 120, 130, 140, 150, 160. The gum base 110 may be comprised of a plurality of ingredients, including any combination of resin, wax and/or elastomer. The base 110 is further easily chewable by the user. In addition, the base 100 may include natural or synthetic substances such as but not limited to: butadiene-styrene rubber, isobutylene-isoprene copolymer (butyl rubber), petroleum wax, petroleum wax synthetic, polyethylene, polyisobutylene polyvinyl acetate, various natural gums, etc. The base 110 may also include a latex or latex blend that includes but is not limited to: leche, caspi, sorva, nispero, tunu, jelutong, chicle, etc. Further, the base 110 may be nicotine-infused.

In differing embodiments, the chew 100 may also contain a natural, plant-derived sweetener 120 such as but not limited to xylitol. The chew 100 may contain other sweeteners 120 such as but not limited to: sugar, dextrose, glucose/corn syrup, erythritol, isomalt, maltitol, mannitol, sorbitol, lactitol, etc. as well as synthetic sweeteners such as but not limited to: aspartame, acesulfame-K, saccharine, sucralose, neohesperidine, dihydrichalcone, etc. Further, the natural, plant-derived sweetener 120 may contain antimicrobial properties and may increase saliva production. Conversely, one embodiment of the chew 100 may be sugar-free and sweetener-free.

The softener/plasticizer agent 130 may include glycerin or vegetable oil that allows the chew 100 to retain moisture and increase flexibility. The chew 100 may also come in a plurality of flavors including but not limited to: tropical, mint, fruit, bubblegum, etc. that are achieved via a flavoring agent 140. The chew 100 may also be any color known in the art as a result of the inclusion of a coloring agent 150.

The chew 100 may also be made of a plurality of edible, food-grade waxes or wax coatings 160 such as but not limited to: synthetic petroleum-based waxes, cane wax, soy wax, bayberry wax, beeswax, carnauba wax, shellac, candelilla wax, sugarcane wax, paraffin wax, canning wax, microcrystalline wax, etc. In addition, the wax coating 160 may contain oils such as but not limited to: wax-based oils, starch-based oils, alginate-based oils, methylcellulose-based oils, zein-based oils, whey-protein based oils, and fatty acid ester-based oils. The coating 160 may also be nicotine-infused.

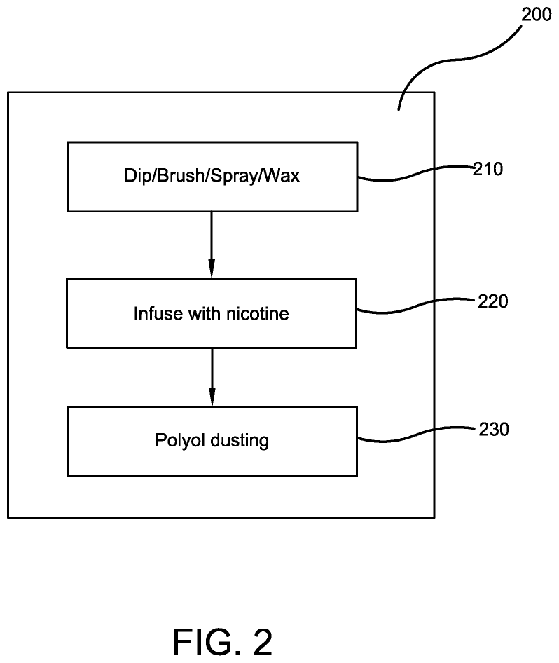

FIG. 2 illustrates a graphical view of a method of applying a wax coating 160 to one potential embodiment of a nicotine chew device for denture users 100 of the present invention in accordance with the disclosed architecture. In the embodiment of the chew 100 that contains a wax coating 160, the coating 160 may be applied via an application method 200. The application method 200 includes dipping, brushing or spraying wax onto the surface of the chew (Block 210). In addition, the wax coating 160 of the chew 100 may be infused with nicotine (Block 220) during the manufacturing process such that chewing the chew 100 releases nicotine. However, the base 110 may have already been infused with nicotine earlier in the manufacturing process, before the wax coating 160 is applied. Therefore, the chew 100 releases nicotine when chewed and can be used as a tool to help users quit smoking. Further, the wax coating 160 may receive a powder dusting of polyol to extend the shelf life of the product (Block 230).

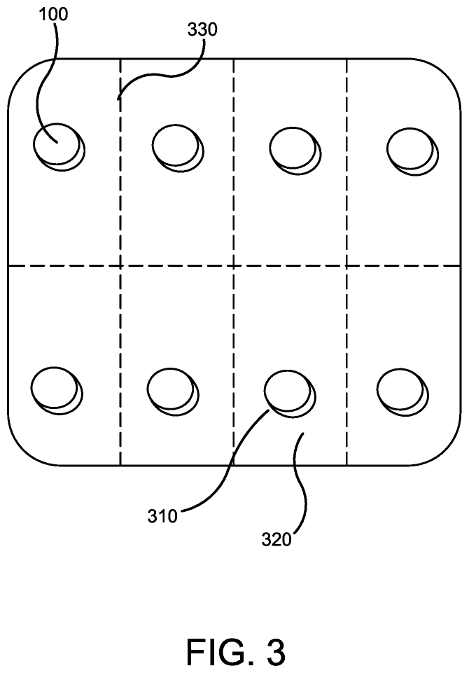

FIG. 3 illustrates a bottom perspective view of one potential embodiment of a nicotine chew device for denture users 100 of the present invention in packaging 300 in accordance with the disclosed architecture. The chew 100 may also come in a packaging 300. In one embodiment, the packaging 300 may contain individually separated compartments 310 that each house a chew 100. Each compartment 310 may further have an individual, peelable cover 320 that seals the chew 100 when it is not being consumed by a user. Further, each compartment 310 may be tear-able along a plurality of perforations 330 to allow a user to separate one compartment 310 and chew 100 from the rest of the packaging.

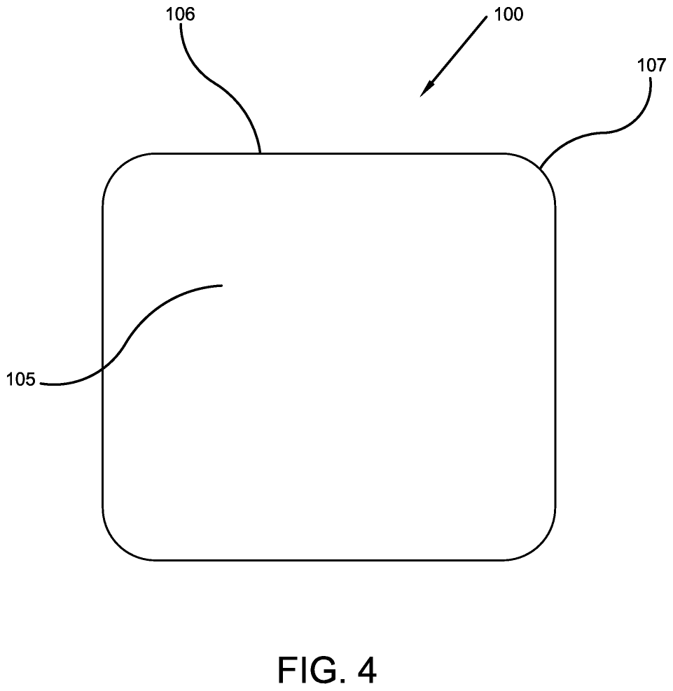

FIG. 4 illustrates a perspective view of one potential embodiment of a nicotine chew device for denture users 100 of the present invention in accordance with the disclosed architecture. It is contemplated the chew 100 may have a body 105 that in differing embodiments may be any shape known in the art such as but not limited to: square, rectangular, circular, oblong, triangular, trapezoidal, octagonal, etc. Further, the body 105 may have rounded edges 106 and rounded corners 107. In differing embodiments, the chew 100 may also be relatively two-dimensional (e.g. thin and flat like a traditional stick of gum known in the art) or relatively three-dimensional and more full in shape.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “nicotine chew device for denture users” and “chew”, are interchangeable and refer to the nicotine chew device for denture users 100 of the present invention.

Notwithstanding the forgoing, the nicotine chew device for denture users 100 of the present invention and its various components can be of any suitable size and configuration as is known in the art without affecting the overall concept of the invention, provided that they accomplish the above-stated objectives. One of ordinary skill in the art will appreciate that the size, configuration and material of the nicotine chew device for denture users 100 as shown in the FIGS. are for illustrative purposes only, and that many other sizes and shapes of the nicotine chew device for denture users 100 are well within the scope of the present disclosure. Although the dimensions of the nicotine chew device for denture users 100 are important design parameters for user convenience, the nicotine chew device for denture users 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A nicotine chew device for a denture wearer, the nicotine chew device comprising:
a base;
a sweetener;
a softener/plasticizer agent;
a flavoring agent;
a coloring agent; and
a wax coating.

2. The nicotine chew device of claim 1, wherein the base is chewable.

3. The nicotine chew device of claim 1, wherein the sweetener is plant-derived.

4. The nicotine chew device of claim 1, wherein the wax coating is an edible wax.

5. A method for applying a wax coating to a nicotine chew device for a denture wearer, the method comprising the steps of:
applying the wax coating to a surface of the nicotine chew device;
infusing a quantity of nicotine into the nicotine chew device; and
dusting the wax coating with a polyol.

6. The method of claim 5, wherein the wax coating is applied via a dipping, a brushing, or a spraying of the nicotine chew device.

7. The method of claim 5, wherein step of infusing the quantity of nicotine is performed before the step of applying the wax coating.

8. The method of claim 5, wherein the quantity of nicotine is infused within the wax coating itself.

9. A nicotine chew device for use by a denture wearer, the nicotine chew device comprising:
a three-dimensional body;
a nicotine-infused chewable base;
a sweetener;
a softener/plasticizer agent;
a flavoring agent;
a coloring agent; and
a wax coating.

10. The nicotine chew device of claim 9, wherein the nicotine-infused chewable base is comprised of any combination of a resin, a wax and an elastomer.

11. The nicotine chew device of claim 9, wherein the nicotine-infused chewable base is comprised of at least one of a butadiene-styrene rubber, a isobutylene-isoprene copolymer (butyl rubber), a petroleum wax, a petroleum wax synthetic, a polyethylene, a polyisobutylene polyvinyl acetate, or a natural gum.

12. The nicotine chew device of claim 9, wherein the nicotine-infused chewable base is comprised of a latex or a latex blend that is comprised of a leche, a caspi, a sorva, a nispero, a tunu, a jelutong or a chicle.

13. The nicotine chew device of claim 9, wherein the sweetener is comprised of at least one of a xylitol, a sugar, a dextrose, a glucose/corn syrup, an erythritol, an isomalt, a maltitol, a mannitol, a sorbitol, a lactitol, an aspartame, an acesulfame-K, a saccharine, a sucralose, a neohesperidine or a dihydrichalcone.

14. The nicotine chew device of claim 9, wherein the sweetener is comprised of an antimicrobial and increases saliva production.

15. The nicotine chew device of claim 9, wherein the wax coating is comprised of a nicotine.

16. The nicotine chew device of claim 9, wherein the softener/plasticizer agent is a glycerin or a vegetable oil.

17. The nicotine chew device of claim 9, wherein the nicotine-infused chewable base is comprised of at least one of a tropical flavor, a mint flavor, a fruit flavor or a bubblegum flavor.

18. The nicotine chew device of claim 9, wherein the wax coating is comprised of at least one of a synthetic petroleum-based wax, a cane wax, a soy wax, a bayberry wax, a beeswax, a carnauba wax, a shellac, a candelilla wax, a sugarcane wax, a paraffin wax, a canning wax or a microcrystalline wax.

19. The nicotine chew device of claim 18, wherein the wax coating is comprised of at least one of a wax-based oil, a starch-based oil, an alginate-based oil, a methylcellulose-based oil, a zein-based oil, a whey-protein based oil or a fatty acid ester-based oil.

20. The nicotine chew device of claim 9, wherein the three dimensional body is a square, a rectangle, a circle, an oblong, a triangle, a trapezoid, or an octagon in shape.
